(19) United States
(12) Patent Application Publication
(10) Pub. No.: US 2009/0008394 A1
(43) Pub. Date: Jan. 8, 2009

(54) Dispenser for animal repellant effluent

(76) Inventor: Charles Colarusso, Brockton, MA (US)

Correspondence Address: John M. Brandt, 60 Thaxter St, Hingham, MA, 02043, US

(21) Appl. No.: 12/214,768

(22) Filed: Jun. 21, 2008

Related U.S. Application Data
(63) Continuation-in-part of application No. 11/174,390, filed on Jul. 5, 2005.
(60) Provisional application No. 60/674,062, filed on Apr. 22, 2005.
(60) Provisional application No. 60/936,915, filed on Jun. 22, 2007.

(51) Int. Cl. B65D 25/14; A61L 9/00; B65D 6/00; B65D 21/00

(52) U.S. Cl. 220/495.06; 239/34; 220/676; 220/23.83

(57) ABSTRACT

A dispenser for animal repellent effluent is disclosed consisting of a chemical repellent containing compartment having a plurality of vents, a compartment cover to protect the repellent from the weather and a positioning or attaching device to fix the dispenser at a selected location. The vents provide for the dissemination of repellent effluent about the location such as a trashcan, dumpster, or exterior of a building. An embodiment in which the dispenser is integrated into and forms a part of the structure of a trash receptacle is also disclosed.

### CROSS REFERENCE TO RELATED APPLICATIONS

This application is based on Provisional Application Ser. No. 60/674,062 filed Apr. 22, 2005 and Ser. No. 60/936,915 filed Jun. 22, 2007, and is a continuation-in-part of Ser. No. 11/174,390 filed Jul. 5, 2005 by the same inventor and is incorporated herein by reference.

### BACKGROUND OF THE INVENTION

1. Field of the Invention

The invention resides in the field of devices for warding off unwanted animals by the release to the environment of an effluent offensive to the animals.

2. Description of the Prior Art

Several devices for emitting a chemical effluent repellent to animals exist in the prior art. The purpose of these is to provide a container for an animal repellent chemical, mothballs for example, which may be situated in an area where animal pests are likely to gather to discourage their presence by an odor offensive to them. Examples are U.S. Pat. Nos. 3,840,145, Almanza; 4,804,142, Riley; 5,379,545, Gall et al.; and 5,988,520, Bitner.

The invention disclosed herein provides unique and novel features over the prior art known to applicant, in particular, the location and secure placement of the dispensers which enhance their use, and the application of the invention to the construction of a trash can.

### SUMMARY OF THE INVENTION

The invention may be summarized as an animal repellent effluent emitter or dispenser which is attachable or securable at a selected location to which animals are likely to be attracted. The apparatus includes a chamber for holding the repellent substance, moth balls for example, a chamber cover for protecting the repellent from the weather, and means for positioning, attaching or securing the dispenser. Vents in the chamber are provided to introduce the repellent effluent into the atmosphere.

Pluralities of embodiments are disclosed herein. The dispenser may be a separate attachable unit or may be molded directly into the structure of a trashcan or dumpster. It may also take the form of a pail with an interior shelf coupled with a stake for holding the dispenser in place by sinking the stake into the ground.

The dispenser may also be integrated directly into the structure of and made a part of a trash receptacle. Both the cover as well as the base or barrel part of the receptacle may be constructed having double or two-ply walls which will provide a means to contain an effluent emitting chemical comprising an animal repellent in a suitable chamber within the walls while isolating the chamber from the environment to prevent the deterioration of the repellent material.

These and other features and advantages, as well as the detailed structure of the invention will become more evident from the description of the preferred embodiment taken in conjunction with the drawings which follows.

### DESCRIPTION OF THE DRAWINGS

FIG. 1 is a cross-sectional view of a first preferred embodiment of the invention;

FIG. 2 is a cross-sectional view of an alternative mounting position of the dispenser of FIG. 1;

FIG. 3 is a perspective view of an additional placement of the dispenser of FIG. 1;

FIG. 4 is a cross-sectional view of an additional mounting arrangement of the dispenser of FIG. 1;

FIG. 5 is a cross-sectional view of an additional embodiment of the invention;

FIG. 6 is a partially cross-sectional front view of another embodiment of the invention;

FIG. 7 is a partially cross-sectional front view of a further embodiment of the invention;

FIG. 8 is a plan view of the embodiment of FIG. 7;

FIG. 9 is a front view of another embodiment of the invention;

FIG. 10 is a cross-sectional side view of an embodiment of the invention comprising a trash receptacle;

FIG. 11 is a front view of one component of the embodiment of FIG. 10;

FIG. 12 is a top view of the same component of the embodiment of FIG. 10; and

FIG. 13 is a top view of a second component of the embodiment of FIG. 10.

### DESCRIPTION OF THE PREFERRED EMBODIMENT

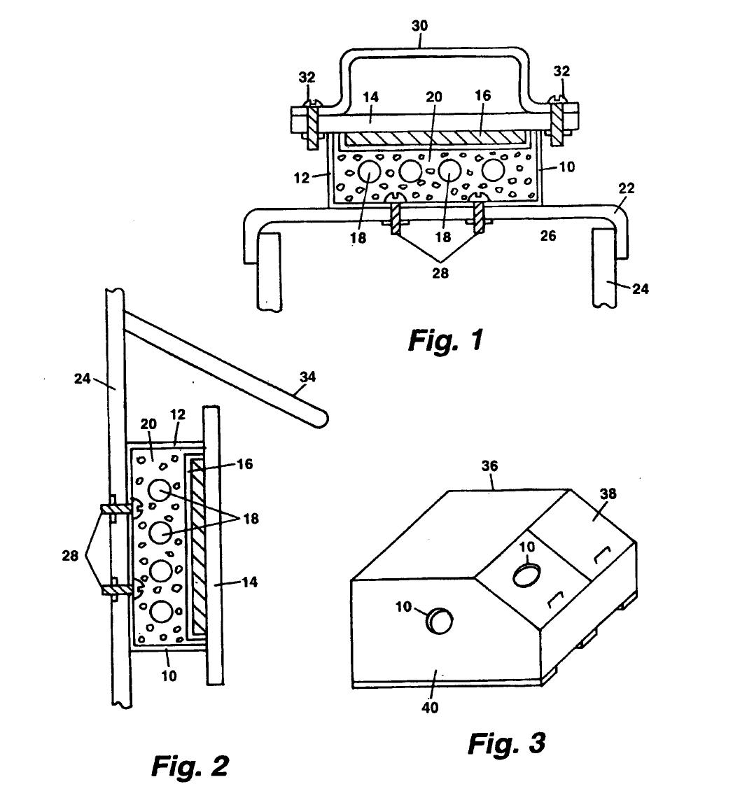

Referring first to FIG. 1, there is shown a cross-sectional view of a first preferred embodiment of the invention in which dispenser 10 of any appropriate shape is composed of a lower compartment 12 for containing a chemical animal repellent and an upper cover 14 for securing and protecting the repellent. The cover may be held in place by the force fit of a flange 16 as shown or by, for example, matching threads, clips, Velcro strips, or similar such mechanical means. Ports 18 are placed about the circumference of compartment 12 to release the vapors from chemical 20, a substance such as moth crystals, disposed in the compartment.

Dispenser 10 is attached to cover 22 of trashcan 24 by, for example, a strong double-sided adhesive tape 26 or other mechanical means such as nut and bolt sets 28. Additionally, a handle 30 may be attached to cover 14 by, for example, nut and bolt sets 32 to lift both dispenser cover 14 and/or trash can cover 22 depending upon how cover 14 is attached to container 12.

Referring next to FIG. 2, the dispenser of FIG. 1 is shown combined with the body of can 24 and in particular is mounted under trash can handle 34 so that, in addition to the protection provided by cover 14, the entire dispenser is shielded from the weather by handle 34 when it is secured in this position.

Referring next to FIG. 3, dispenser 10 is illustrated mounted on dumpster 36 on both door 38 and side 40. The same means as described above may be used to secure the dispenser to the dumpster.

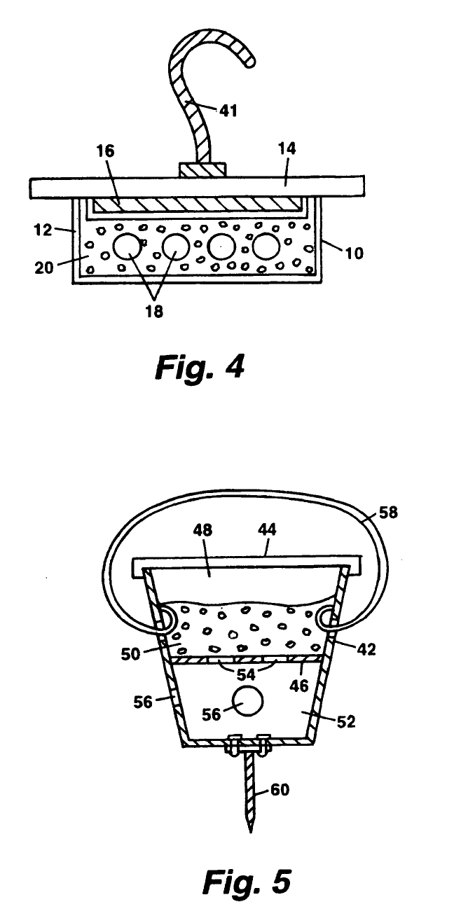

FIG. 4 illustrates an alternative means of positioning dispenser 10 by providing hook 41 for locating the invention in a general area where animals are unwanted such as a borough in a yard or near a house foundation.

FIG. 5 is a cross-sectional view of another embodiment of the invention showing a dispenser comprising a pail 42 having a cover 44 and an interior perforated shelf 46. Upper chamber 48 holds an animal repellent chemical 50 and lower chamber 52 further isolates the chemical from the environment. Perforations 54 in shelf 46 and ports 56 in chamber 52 allow chemical effluent generated by chemical 50 to escape into the surrounding atmosphere. Handle 58 and/or ground stake 60 are provided to move and secure the dispenser at a desired location.

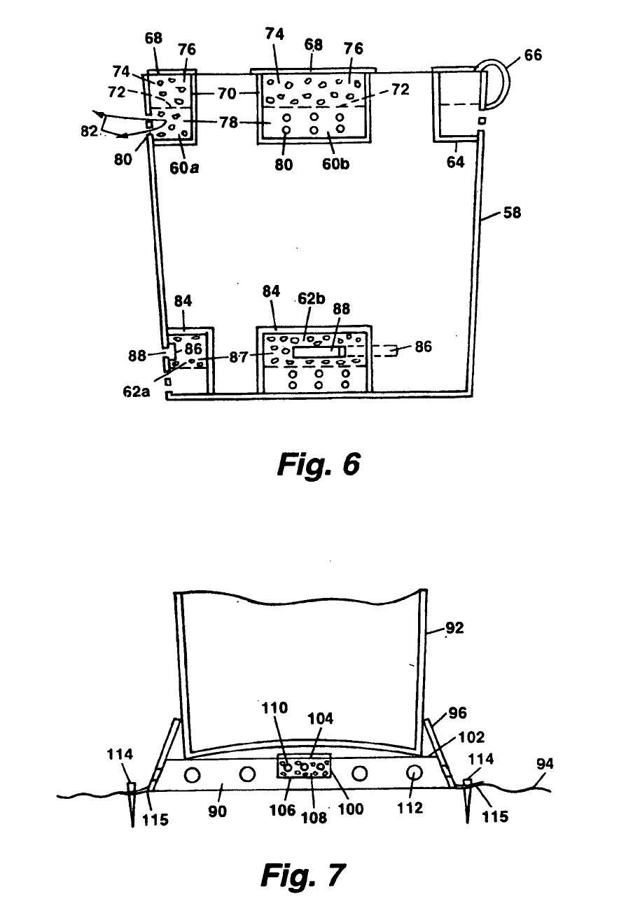

Referring next to FIG. 6, a partially cross-sectional front view of an additional embodiment of the invention is shown in which three variations of a dispenser are disposed in a trashcan or receptacle 58. The cover of can 58 is not shown for purposes of clarity. Two of the dispenser variations, 60 and 62 are molded directly into can 58 and the third dispenser variation 64 is a separate unit and is suspended from the rim of the can by, for example, a wire clip 66.

Dispenser 60 is shown twice as a side view 60a and as a front view 60b and is composed of cover 68 and dispenser body 70 which cover may either snap on and off or slide back and forth to open and close the interior of dispenser body 70. Perforated shelf 72 divides the dispenser into an upper chamber 74 for containing repellent chemical 76 and a lower chamber 78. Ports 80 formed directly in the wall of can 80 allow the repellent effluent, as indicated by arrow 82, to directly enter the atmosphere.

The second variation 62 is also illustrated as two views, as a side view 62a and a front view 62b, and is the same as dispenser 60 except that the cover 84 is not removable. Instead sliding door 86 for the insertion of chemical 87 is arranged to cover port 88 formed directly in the wall of the can.

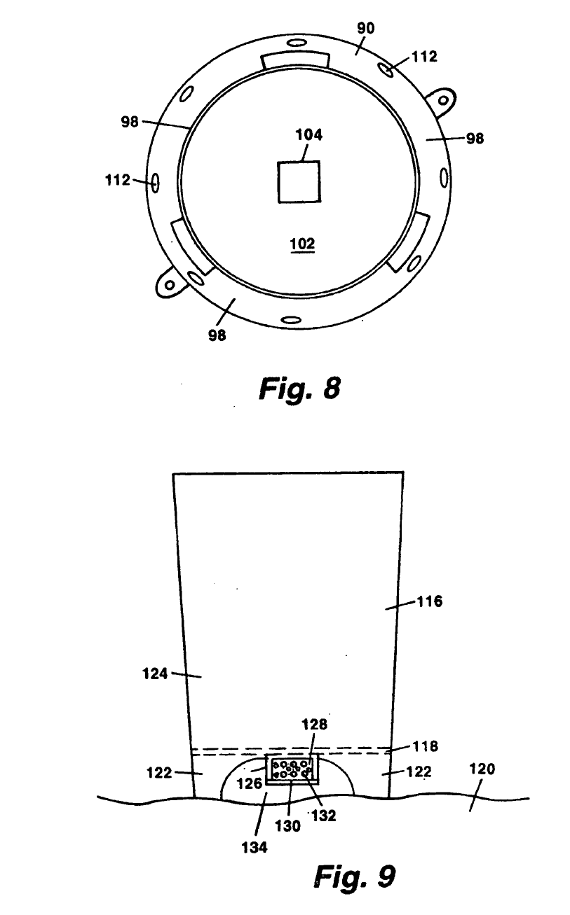

Referring next to FIGS. 7 and 8, a cross-sectional side view and plan view of an additional embodiment of the invention is illustrated. This arrangement comprises a base 90 for supporting a trashcan 92 above ground level 94. Wall 96 extending above base 90 may be added to aid in preventing can 92 from tipping over and may completely surround the can or may be a partial wall disposed in sections 98 about the base to achieve the same purpose.

Dispenser 100 is disposed in top 102 of base 90 and includes removable cover 104 and chamber 106 for containing animal repellent chemical 108. Ports 110 are disposed in chamber 106 and ports 112 are disposed in base 90 to allow the effluent from repellent 108 to disperse to the environment. Base 90 may be held in place by the weight of can 92 alone or may additionally be secured by stakes 114 driven into ground 94 through tabs 114 attached to base 90.

FIG. 9 illustrates a front view of a further embodiment of the invention in which trashcan 116 has a bottom 118 raised above ground 120 by extensions 122 of sidewall 124. Compartment 126 for containing animal repellent chemical 128 is mounted below bottom 118 and has removable cover 130 for inserting chemical 128. Ports 132 allow the chemical effluent to pass into the environment through space 134 at the bottom of the can as created by extensions 122.

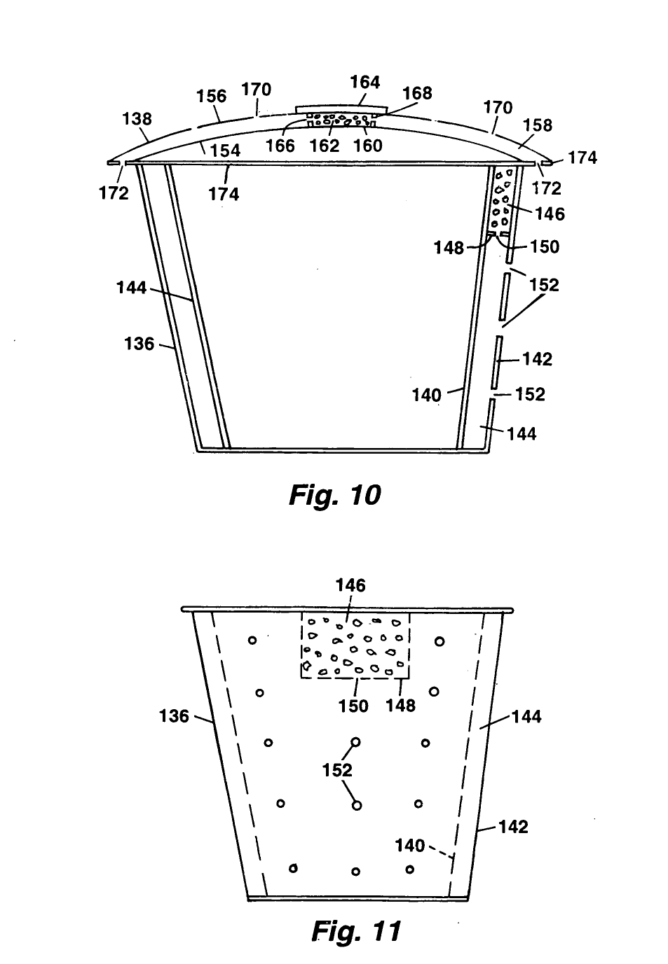

Referring next to FIGS. 10-13, an additional embodiment of the invention is shown in which a trash receptacle has an inner and outer or two-ply wall. Either the barrel portion or cover may be constructed with this design to accomplish the purpose of the invention. FIG. 10 is a cross-sectional side view of an entire receptacle composed of barrel 136 and cover 138. Barrel 136 has an inner wall 140 for containing trash and an outer wall 142 which together form an effluent emitting chamber 144.

Compartment 146 is disposed in the top portion of barrel 136 for the purpose of containing a material which emits an effluent offensive to animals as described above. A bottom shelf 148 is provided which has one or more ports 150 which communicate with effluent emitting chamber 144.

Ports 152 are disposed in outer wall 142 communicating with the environment thus providing an effluent path from compartment 146 to chamber 144 to the environment. Ports 150 and 152 are spaced apart from one another a sufficient distance to prevent the effluent emitting material in compartment 146 from direct exposure from outside moisture and adverse weather such as rain, snow, and the like. Chamber 144 in the side of the barrel therefore forms an intermediary baffle to keep the weather and also children away from the chemical while allowing passage of the animal repelling effluent.

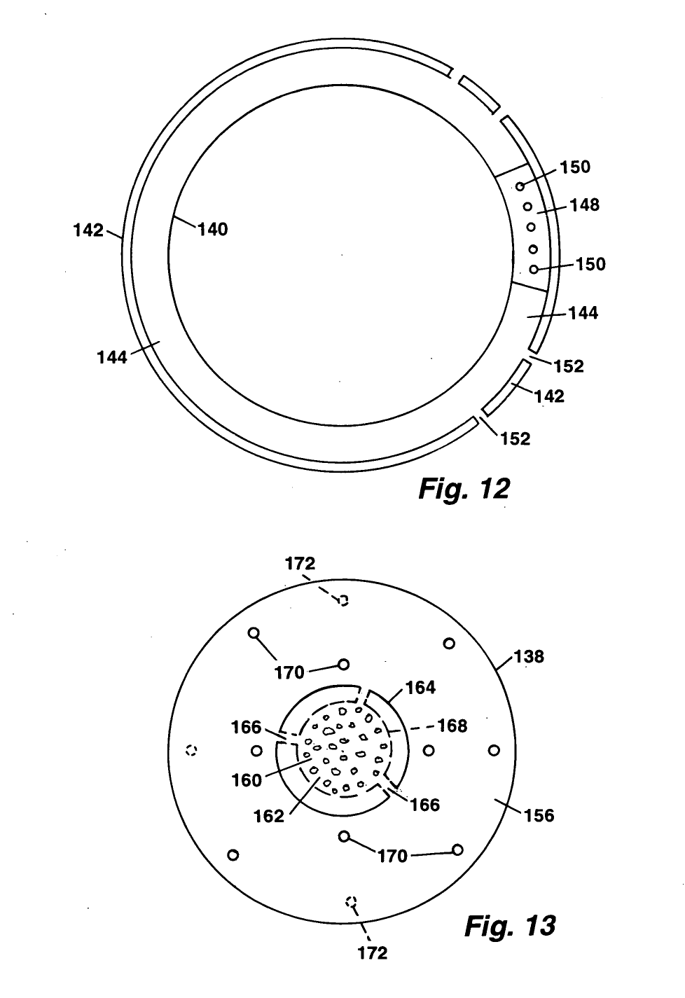

The above described structure of the barrel portion of the receptacle is further illustrated in a front view, FIG. 11, and a top view, FIG. 12, wherein like reference numerals refer to like components of FIG. 10.

FIG. 10 also illustrates a cover portion 138 which may be used alone with single wall barrel or as shown, with a two-ply barrel of the invention. Cover 138 may similarly be constructed of a two-ply wall shown as inner wall 154 and outer wall 156 forming an effluent chamber 158. Compartment 160 is disposed in the center of cover member 138 for the purpose of containing a material 162 which emits an animal repellent effluent. A separate compartment cover is provided to secure and protect the material from the weather, particularly moisture in the environment such as rain or snow.

Ports 166 are placed in sidewall 168 to provide for passage of effluent to chamber 158. Ports 170 are disposed about outer wall 156 to provide effluent release to the environment. Additional ports 172 communicating with chamber 158 are placed about the lower rim 174 of the cover to provide an additional effluent path and further to provide drainage for water from rain or snow entering chamber 158 through ports 170. Such drainage water will also assist in the downward disposition of the effluent at such times as it is present from storms or snow fall.

As variations in the above-described embodiments will now be obvious to those skilled in the art, the invention is accordingly defined by the following claims.

What is claimed is:

1. In an outdoor trash receptacle comprising a barrel member for containing said trash and a cover member adapted to fit over said barrel member to seal the interior of said barrel member from the environment wherein each of said barrel and cover members has a first inner wall, the improvement which comprises animal repellent effluent emitting means integrated into the structure of said receptacle, said means comprising:
A. a second outer wall surrounding at least a portion of said inner wall in at least one of said barrel and cover members forming an effluent emitting chamber, said second wall having a first set of vents communicating with the exterior of said wall for emitting said effluent to the environment; and
B. a container disposed between said first and second walls for containing a chemical material arranged to emit an animal repellant effluent, said container having a second set of vents communicating with said effluent emitting chamber, said first and second sets of vents disposed in spaced apart relationship.

2. In an outdoor trash receptacle comprising a barrel member for containing said trash and a cover member adapted to fit over said barrel member to seal the interior of said barrel member from the environment wherein said barrel and member has a first inner wall, the improvement which comprises animal repellent effluent emitting means integrated into the structure of said barrel member, said means comprising:
A. a second outer wall surrounding at least a portion of said inner wall forming an effluent emitting chamber, said second wall having a first set of vents communicating with the exterior of said wall for emitting said effluent to the environment; and
B. a container disposed between said first and second walls for containing a chemical material arranged to emit an animal repellant effluent, said container having a second set of vents communicating with said effluent emitting chamber, said first and second sets of vents disposed in spaced apart relationship.

3. In an outdoor trash receptacle comprising a barrel member for containing said trash and a cover member adapted to fit over said barrel member to seal the interior of said barrel member from the environment wherein said cover member has a first inner wall, the improvement which comprises animal repellent effluent emitting means integrated into the structure of said receptacle, said means comprising:
A. a second outer wall surrounding at least a portion of said inner wall forming an effluent emitting chamber, said second wall having a first set of vents communicating with the exterior of said wall for emitting said effluent to the environment; and
B. a container disposed between said first and second walls for containing a chemical material arranged to emit an animal repellant effluent, said container having a second set of vents communicating with said effluent emitting chamber, said first and second sets of vents disposed in spaced apart relationship.

4. The cover member of claim 3 wherein said inner wall of said cover member has a bottom rim extending outwardly to said second wall and said rim has at least one port communicating with said effluent emitting chamber for draining water from said chamber.

5. The cover member of claim 3 wherein said container has a cover disposed in said outer wall providing access to the interior of said container.
